(19) United States
(12) Patent Application Publication
(10) Pub. No.: US 2023/0115577 A1
(43) Pub. Date: Apr. 13, 2023

(54) MEDICAL IMAGE PROCESSING APPARATUS, MEDICAL IMAGE PROCESSING METHOD, AND STORAGE MEDIUM

(71) Applicant: CANON KABUSHIKI KAISHA, Tokyo (JP)

(72) Inventors: Kazuma Obara, Tokyo (JP); Daisuke Yamada, Kanagawa (JP)

(21) Appl. No.: 18/045,240

(22) Filed: Oct. 10, 2022

(30) Foreign Application Priority Data
Oct. 12, 2021 (JP) 2021-167597

(51) Int. Cl. A61B 6/00

(52) U.S. Cl. CPC A61B 6/5205; A61B 6/5217

(57) ABSTRACT

A medical image processing apparatus includes a processing unit configured to process, using learning result data that is at least one or more pieces of learning result data obtained by machine learning and is selected based on imaging information, a medical image obtained based on the imaging information, the imaging information including information regarding a radiation detection apparatus used for imaging.

### BACKGROUND

### Field of the Disclosure

The present disclosure relates to a medical image processing apparatus, a medical image processing method, and a storage medium.

### Description of the Related Art

Radiation imaging apparatuses that use flat panel detectors (FPDs) configured with semiconductor materials are widely used in medical image diagnosis and non-destructive inspection. The radiation imaging apparatuses have functions of performing image processing suitable for diagnoses such as noise elimination on images captured using the FPDs. Some of the image processing use machine learning.

Japanese Patent Application Laid-Open No. 2020-92976 discusses a method of selectively obtaining learning data associated with examination information of a processing target image, and performing an inference in machine learning.

### SUMMARY

According to an aspect of the present disclosure, a medical image processing apparatus includes a processing unit configured to process, using learning result data that is at least one or more pieces of learning result data obtained by machine learning and is selected based on imaging information, a medical image obtained based on the imaging information, the imaging information including information regarding a radiation detection apparatus used for imaging.

Further features of various embodiments will become apparent from the following description of exemplary embodiments with reference to the attached drawings.

### BRIEF DESCRIPTION OF THE DRAWINGS

FIG. 1 illustrates a configuration example of a radiation imaging system according to a first exemplary embodiment.

FIG. 2 is a flowchart illustrating imaging processing according to the first exemplary embodiment.

FIG. 3 is a flowchart illustrating obtaining processing of learning result data according to the first exemplary embodiment.

FIG. 4 is a flowchart illustrating transfer output processing according to a second exemplary embodiment.

FIG. 5 illustrates a configuration example of a radiation imaging system according to a third exemplary embodiment.

### DESCRIPTION OF THE EMBODIMENTS

In noise reduction processing using machine learning, accuracy can be improved by selectively obtaining a learned parameter suitable for a noise characteristic and using it for an inference in the machine learning. The noise characteristic depends on the type of a radiation detection apparatus used for imaging and a preprocessing content of a filter and the like with respect to a processing target image. Examination information used in a conventional technique does not include the above-described information, and the learned parameter suitable for the noise characteristic cannot be selected. Alternatively, even in a case where irradiation field recognition is performed on an image, accuracy of the recognition can be improved by considering the type of the radiation detection apparatus used for imaging and the preprocessing content of the filter and the like with respect to the processing target image.

Thus, one aspect of a present exemplary embodiment is directed to a technique of performing a highly accurate inference by selecting a learned parameter using imaging information, such as information about a radiation detection apparatus and information about a processing target image.

According to the present exemplary embodiment, a highly accurate inference can be performed by selecting a learned parameter using imaging information.

The exemplary embodiments of the present disclosure will be described below with reference to the attached drawings. The exemplary embodiments which will be described below do not restrict every embodiment according to the claims. All of combinations of the features described in the exemplary embodiments are not always essential to the means for solution according to the present disclosure.

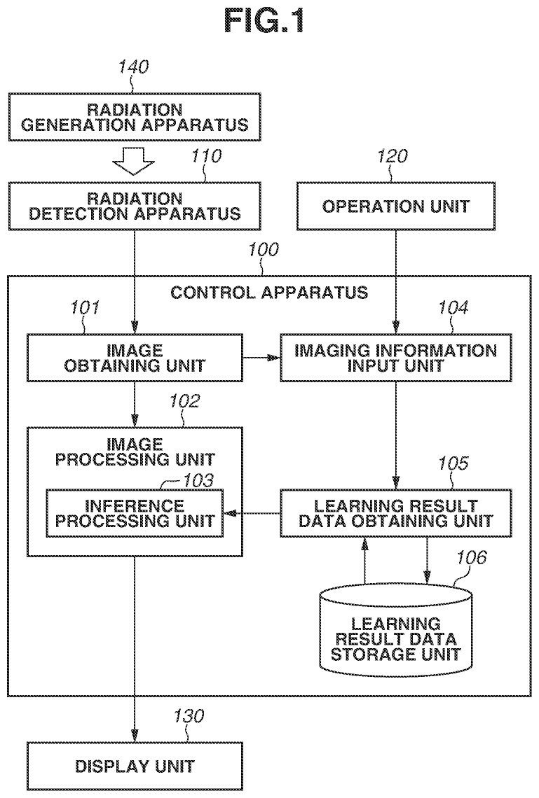

A configuration and an operation of a radiation imaging system according to the exemplary embodiment of the present disclosure are described with reference to FIGS. 1 to 3.

FIG. 1 illustrates a configuration example of a radiation imaging system according to a first exemplary embodiment. The radiation imaging system includes a control apparatus 100, a radiation detection apparatus 110, an operation unit 120, a radiology department information system, a display unit 130, and a radiation generation apparatus 140. The control apparatus 100 controls radiation imaging using the radiation detection apparatus 110 and the radiation generation apparatus 140.

The radiation detection apparatus 110 detects radiation that has emitted from the radiation generation apparatus 140 and transmitted through an examinee (not illustrated), and outputs image data corresponding to the radiation. Image data can be rephrased as a medical image and a radiographic image. Specifically, the radiation detection apparatus 110 detects the radiation transmitted through the examinee as an electric charge corresponding to a transmitted radiation dose. For example, in the radiation detection apparatus 110, a direct conversion type sensor that directly converts radiation into an electric charge using amorphous selenium (a-Se) or the like that converts radiation into an electric charge and an indirect type sensor that uses a scintillator, such as cesium iodide (CsI), that converts radiation into visible light and a photoelectric conversion element, such as amorphous silicon (a-Si), may be used. Further, the radiation detection apparatus 110 preforms analog-to-digital (A/D) conversion on the detected electric charge to generate image data and outputs the image data to the control apparatus 100.

The control apparatus 100 is connected to the radiation detection apparatus 110 by, for example, a wired or wireless network or a dedicated line. The radiation detection apparatus 110 captures radiation generated by the radiation generation apparatus 140 and outputs the image data to the control apparatus 100. The control apparatus 100 has an application function that operates in a computer. Specifically, the control apparatus 100 includes one or more processors and memories, and the processor executes computer-executable instructions (e.g., one or more programs) stored in the memories and thus realizes each function unit described below. However, a part or all of each function unit may be realized by dedicated hardware. The control apparatus 100 performs image processing on the image data output from the radiation detection apparatus 110 to generate an image and displays the image on the display unit 130. The operation unit 120 receives an instruction from an operator. The control apparatus 100 also has a function of controlling each component. The control apparatus 100 outputs an image to the display unit 130 or provides a graphical user interface using the display unit 130 while controlling an operation of the radiation detection apparatus 110.

The control apparatus 100 controls timing at which the radiation generation apparatus 140 generates radiation and an imaging condition of the radiation. In the control apparatus 100, an image obtaining unit 101 controls timing at which the radiation detection apparatus 110 captures image data and timing at which the radiation detection apparatus 110 outputs the image data. An imaging information input unit 104 is an example of a first obtaining unit that obtains imaging information. The imaging information input unit 104 according to the present exemplary embodiment receives the imaging information manually input by the operator from the operation unit 120 or obtains the imaging information from the image obtaining unit 101 and causes a user to select the imaging information using the operation unit 120. The imaging information input to the imaging information input unit 104 is managed in association with the image data captured by the radiation detection apparatus 110.

A learning result data obtaining unit 105 reads out learning result data from a learning result data storage unit 106. The learning result data storage unit 106 stores learning result data that is obtained by machine learning using a teacher image. The learning result data storage unit 106 also stores correspondence information that indicates correspondence between the imaging information and the learning result data to be read out. The learning result data obtaining unit 105 also reads out information that associates various words and a combination of words, which are included in the imaging information, with the learning result data stored in the learning result data storage unit 106. Accordingly, the learning result data obtaining unit 105 can obtain the learning result data to be used for processing by an image processing unit 102 (an inference processing unit 103) based on the word included in the imaging information. Further, the learning result data obtaining unit 105 can read out the learning result data corresponding to the selected imaging information from the learning result data storage unit 106 by referring to the correspondence information. Specifically, the learning result data obtaining unit 105 is an example of a readout unit that reads out the learning result data selected based on the above-described imaging information from a storage unit (the learning result data storage unit 106) that stores the learning result data obtained in advance by machine learning.

The image obtaining unit 101 is an example of a second obtaining unit that obtains a medical image that is obtained based on the imaging information obtained by the first obtaining unit (the imaging information input unit 104). According to the present exemplary embodiment, a radiographic image captured by the radiation detection apparatus 110 is obtained as a medical image. The image processing unit 102 performs image processing, such as contrast adjustment, on the image data output from the radiation detection apparatus 110. The image processing unit 102 can also perform image processing, such as trimming and rotation, on the image output from the radiation detection apparatus 110. The inference processing unit 103 performs inference processing using the learning result data by machine learning, such as noise reduction. The image processing unit 102 may include a plurality of inference processing units as the inference processing unit 103 corresponding to purposes such as irradiation field recognition and gradation processing in addition to noise reduction. The image processing unit 102 displays the image after image processing on the display unit 130. The image processing unit 102 and the inference processing unit 103 are examples of a processing unit that performs processing on the obtained medical image using the learning result data read out by the readout unit (the learning result data obtaining unit 105).

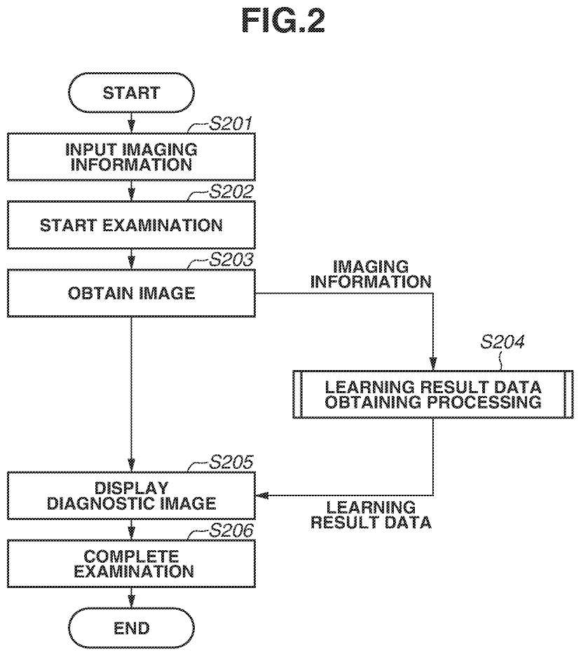

Next, radiographic image processing according to the first exemplary embodiment is described with reference to a flowchart in FIG. 2.

In step S201, the imaging information input unit 104 causes a user to select one of a plurality of pieces of imaging information obtained from the operation unit 120 and sets the selected one as an examination target. The above-described processing can be realized in such a manner that, for example, the plurality of pieces of obtained imaging information is displayed in a list format, and in response to an operation input by a user to select one of the imaging information from the list, the selected imaging information is set as the examination target. The user may directly input the imaging information from the operation unit 120.

In step S202, the control apparatus 100 starts an examination by transmitting to the radiation detection apparatus 110 a signal for shifting it to a preparation state according to the set imaging information.

In response to the signal, for example, the radiation detection apparatus 110 controls a bias power supply by a main control circuit and applies a bias voltage to a two-dimensional image capturing element. Subsequently, the radiation detection apparatus 110 performs initialization to read out an image signal from a pixel array by a drive circuit in order to read out a dark current signal accumulated in a pixel. After the initialization is completed, the radiation detection apparatus 110 transmits status information indicating a state ready for obtaining a radiographic image to the control apparatus 100. In addition, the control apparatus 100 (the imaging information input unit 104) sets an operation parameter (a tube voltage and the like) of the radiation generation apparatus 140 based on the imaging information selected in step S201. Upon receiving a notification that the imaging preparation is completed by the status information from the radiation detection apparatus 110, the control apparatus 100 notifies the radiation generation apparatus 140 of an exposure permission.

In step S203, the image obtaining unit 101 obtains a radiographic image captured by the radiation detection apparatus 110. More specifically, for example, if the radiation generation apparatus 140 notified of the exposure permission emits radiation in response to an operation of an exposure button, the drive circuit of the radiation detection apparatus 110 reads out an image signal obtained by detecting the emitted radiation by a readout circuit and generates a radiographic image. The radiation detection apparatus 110 transmits the generated radiographic image to the control apparatus 100. The image obtaining unit 101 of the control apparatus 100 receives the radiographic image. Thus, the imaging information input unit 104 and the image obtaining unit 101 of the control apparatus 100 function as a control unit that controls an operation of radiation imaging based on the imaging information and obtains a radiographic image obtained by the radiation imaging as a radiographic image captured based on the imaging information.

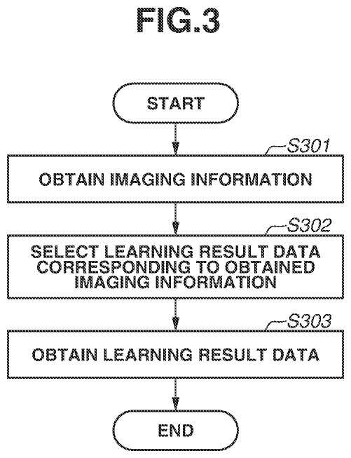

Meanwhile, in step S204, the learning result data obtaining unit 105 selectively obtains the learning result data from the learning result data storage unit 106 based on the imaging information selected in step S203. The processing in step S204 is described in detail below with reference to a flowchart in FIG. 3. The imaging information includes, for example, information regarding the radiation detection apparatus and information regarding the processing target image. For example, the information regarding the radiation detection apparatus includes information regarding a fluorescence substance included in the radiation detection apparatus, and an imaging resolution, a pixel pitch, and a drive mode of the radiation detection apparatus. The information regarding the processing target image includes a size and a use application of the image and information regarding preprocessing. For example, the size of the image can be expressed as a full size, ⅛, ¼, ½, and the like, and the use application of the image can be expressed as a preview, transfer output, or the like. In some embodiments, the information regarding the processing target image includes information about a ⅛ thinned-out preview image, a ¼ thinned-out preview image, a ½ thinned-out preview image, a full size image without thinning out, and a transfer output image. The imaging information includes at least one of the information regarding the radiation detection apparatus used for imaging and the information regarding the processing target image. The processing in step S204 can be executed in parallel with the processing in step S203 (obtaining of the radiographic image).

In step S205, the image processing unit 102 executes image processing on the radiographic image obtained by the image obtaining unit 101. At that time, the inference processing unit 103 executes inference processing by machine learning using the learning result data obtained in step S204. The inference processing unit 103 performs, for example, noise reduction processing on the image, annotation processing to determine a display content such as an annotation to be superimposed on the image, or gradation processing. In diagnostic image processing, information from which a result of the above-described processing or the selected learning result data can be determined may be displayed on the display unit 130.

In step S206, the control apparatus 100 completes the examination in response to an input operation by the operator.

Next, the obtaining processing of the learning result data (the processing in step S204) by the learning result data obtaining unit 105 is described with reference to the flowchart in FIG. 3.

In step S301, the learning result data obtaining unit 105 obtains the imaging information determined in step S203.

In step S302, the learning result data obtaining unit 105 selects the learning result data corresponding to the imaging information obtained in step S301.

Then, in step S303, the learning result data obtaining unit 105 reads out the learning result data selected in step S302 from the learning result data storage unit 106, loads the read learning result data so that it can be used by, for example, the inference processing unit 103, and stores the loaded learning result data in the storage unit, which is not illustrated.

As described above, according to the first exemplary embodiment, the learning result data corresponding to the imaging information is selectively obtained from the learning result data storage unit 106, so that a highly accurate inference can be performed. In addition, the image processing unit 102 can realize noise reduction processing using optimum learning result data. Accordingly, desired medical image processing is performed, and a diagnostic performance is improved.

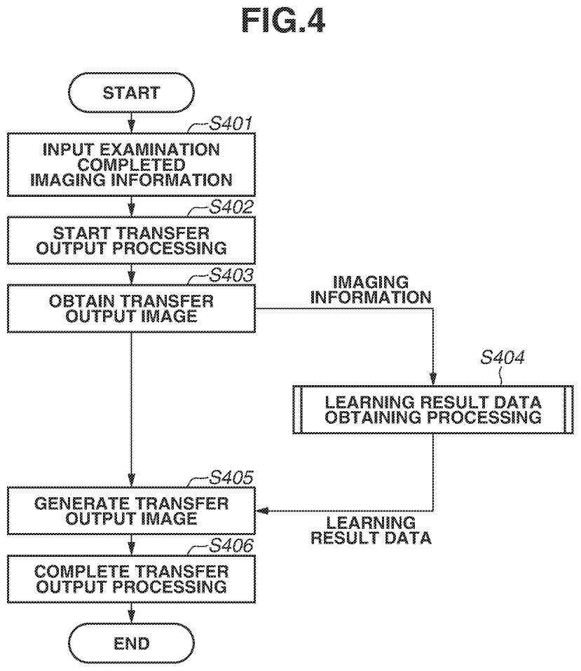

According to the first exemplary embodiment, the flow at the time of imaging is described, but a medical image captured by a control apparatus is generally output to an external device for image confirmation connected to an in-hospital network and is used for a diagnosis and the like. According to a second exemplary embodiment, a flow in a case where a medical image is output by an external device, such as a picture archiving and communication system (PACS) or a printer, in a state where there is the examination completed according to the first exemplary embodiment is described. Processing procedures executed by a radiation imaging system according to the second exemplary embodiment are described with reference to a flowchart in FIG. 4.

In step S401, the imaging information input unit 104 causes a user to select one of a plurality of pieces of examination completed imaging information obtained from the operation unit 120 and sets the selected one as a transfer output target.

The above-described processing can be realized in such a manner that, for example, the plurality of pieces of obtained examination completed imaging information is displayed in a list format, and in response to an operation input by a user to select one piece of the examination completed imaging information from the list, the selected examination completed imaging information is set as the transfer output target. The user may directly input the examination completed imaging information from the operation unit 120.

In step S402, the operation unit 120 starts transfer output processing of the examination completed imaging information selected by the user.

In step S403, the image obtaining unit 101 obtains a radiographic image associated with the examination completed imaging information from a non-illustrated image storage unit.

Meanwhile, in step S404, the learning result data obtaining unit 105 selectively obtains the learning result data from the learning result data storage unit 106 based on the imaging information selected in step S403.

In step S405, the image processing unit 102 executes transfer output image processing on the radiographic image obtained by the image obtaining unit 101. At that time, the inference processing unit 103 executes inference processing by machine learning using the learning result data obtained in step S404. The inference processing unit 103 performs, for example, noise reduction processing on the transfer output image and annotation processing to determine a display content, such as an annotation to be superimposed on the transfer output image. In the transfer output, it is assumed that image processing different from that at the time of preview is executed due to a difference in a use application, such as image confirmation being performed on a screen of a high definition monitor or the like, at an output destination. Thus, the learning result data can be used differently in such a manner that, the learning result data that prioritizes speed is used in a preview, and the learning result data that prioritizes image quality is used in the transfer output.

In step S406, the control apparatus 100 completes the transfer output processing.

As described above, according to the second exemplary embodiment, a highly accurate inference can be performed using optimum learning result data suitable for transfer processing even in the transfer processing. In addition, noise reduction processing can be realized using the optimum learning result data.

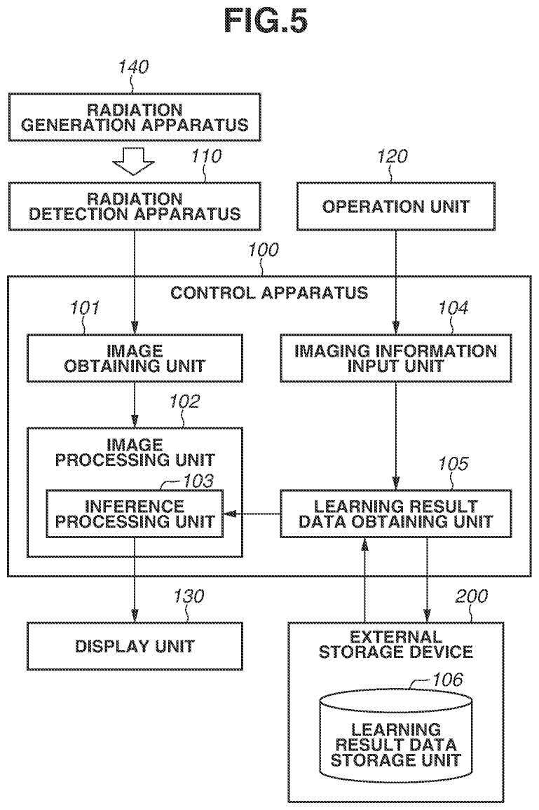

According to the first exemplary embodiment, the configuration in which the control apparatus 100 includes the learning result data storage unit 106 (FIG. 1) is described, but some embodiments are not limited to this configuration. An external storage device that is communicably connected to the control apparatus 100 may include the learning result data storage unit 106. FIG. 5 is a block diagram illustrating a configuration example of a radiation imaging system according to a third exemplary embodiment. In FIG. 5, the learning result data storage unit 106 is arranged in an external storage device 200 provided outside the control apparatus 100. In FIG. 5, components similar to those according to the first exemplary embodiment are denoted by the same reference numerals.

Examples of the external storage device 200 include a network storage, an external computer, and a cloud.

The control apparatus 100 needs to communicate with the external storage device 200 to obtain the learning result data, and a form of communication may be any form such as wired or wireless.

According to the above-described third exemplary embodiment, as in the first exemplary embodiment, the learning result data corresponding to the imaging information is selectively obtained, so that operability of a medical image processing apparatus can be improved. Further, according to the third exemplary embodiment, the learning result data can be shared with the control apparatus 100 in another facility, so that the latest learning result data can be easily distributed and managed.

According to the first exemplary embodiment, obtaining of the learning result data in noise reduction processing using the imaging information is described. However, the image processing unit 102 may perform image processing using other machine learning as long as learning result data using information included in at least one piece of imaging information is obtained. For example, a medical region of a subject that is frequently imaged is different depending on a size of the radiation detection apparatus. Thus, the learning result data obtaining unit 105 obtains the learning result data depending on the size of the radiation detection apparatus and is expected to improve accuracy of irradiation field recognition processing using machine learning.

According to a fourth exemplary embodiment as described above, the learning result data corresponding to the imaging information is selectively obtained, so that operability of a medical image processing apparatus can be improved as in the first exemplary embodiment.

According to the first exemplary embodiment, obtaining of the learning result data using the imaging information is described. However, not only a weight parameter but also information regarding a structure of a neural network to be used in an inference in machine learning may be obtained. For example, the learning result data storage unit 106 stores a first neural network that prioritizes speed to shorten an inference processing time and a second neural network that prioritizes image quality. At that time, in a case where a processing target image is a preview image that prioritizes display speed, the learning result data obtaining unit 105 obtains the learning result data including information regarding a structure of the first neural network. In a case of a transfer output image that prioritizes image quality, the learning result data obtaining unit 105 obtains the learning result data including information regarding a structure of the second neural network.

According to a fifth exemplary embodiment as described above, the information regarding the structure of the neural network corresponding to the imaging information is selectively obtained, so that a highly accurate inference can be performed as in the first exemplary embodiment. In addition, operability of a medical image processing apparatus can be improved.

### OTHER EMBODIMENTS

Some embodiment(s) can also be realized by a computer of a system or apparatus that reads out and executes computer-executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer-executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer-executable instructions. The computer-executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present disclosure has described exemplary embodiments, it is to be understood that some embodiments are not limited to the disclosed exemplary embodiments. The scope of the following claims is to be accorded the broadest interpretation so as to encompass all such modifications and equivalent structures and functions.

This application claims priority to Japanese Patent Application No. 2021-167597, which was filed on Oct. 12, 2021 and which is hereby incorporated by reference herein in its entirety.

What is claimed is:

1. A medical image processing apparatus comprising:
a readout unit configured to read out, from a storage unit that stores at least one or more pieces of learning result data obtained by machine learning, learning result data selected based on imaging information that includes information regarding a radiation detection apparatus used for imaging; and
a processing unit configured to process a medical image obtained based on the imaging information using the learning result data read out by the readout unit.

2. A medical image processing apparatus comprising:
a processing unit configured to process, using learning result data that is at least one or more pieces of learning result data obtained by machine learning and is selected based on imaging information, a medical image obtained based on the imaging information, the imaging information including information regarding a radiation detection apparatus used for imaging.

3. The medical image processing apparatus according to claim 1, wherein the information regarding the radiation detection apparatus includes information regarding a fluorescence substance included in the radiation detection apparatus.

4. The medical image processing apparatus according to claim 1, wherein the information regarding the radiation detection apparatus includes at least one piece of information of an imaging resolution and a pixel pitch of the radiation detection apparatus.

5. The medical image processing apparatus according to claim 1,
wherein the imaging information further includes information regarding a processing target image, and
wherein the information regarding the processing target image includes at least one piece of information of a size of an image, a use application of the image, and information regarding preprocessing.

6. The medical image processing apparatus according to claim 1, wherein the readout unit reads out learning result data from the storage unit that is externally provided.

7. The medical image processing apparatus according to claim 1, further comprising an input unit configured to cause a user to select one of a plurality of pieces of imaging information that includes the information regarding the radiation detection apparatus used for imaging,
wherein the imaging information selected by the input unit is obtained.

8. The medical image processing apparatus according to claim 7,
wherein the storage unit stores correspondence information that indicates correspondence between imaging information and learning result data to be read out for each of the plurality of pieces of imaging information, and
wherein the readout unit reads out the learning result data corresponding to the selected imaging information from the storage unit with reference to the correspondence information.

9. The medical image processing apparatus according to claim 1,
wherein the storage unit further stores information regarding a structure of a neural network, and
wherein the readout unit reads out the learning result data and the information regarding the structure of the neural network based on the imaging information.

10. The medical image processing apparatus according to claim 1, wherein the processing unit performs at least one processing of noise reduction processing, irradiation field recognition processing, annotation processing, and gradation processing using the learning result data read out by the readout unit.

11. A radiation imaging system in which a radiation detection apparatus used for imaging and the medical image processing apparatus according to claim 1 are communicably connected to each other.

12. A method for processing a medical image, the method comprising:
processing a medical image obtained based on imaging information that includes information regarding a radiation detection apparatus used for imaging using learning result data that is at least one or more pieces of learning result data obtained by machine learning and is selected based on the imaging information.

13. A non-transitory computer-readable storage medium storing a program for causing a computer to execute the method according to claim 12.
